(19) United States
(12) Patent Application Publication
(10) Pub. No.: US 2019/0100585 A1
(43) Pub. Date: Apr. 4, 2019

(54) COMPOSITIONS AND METHODS FOR TREATING RHEUMATOID ARTHRITIS

(71) Applicants: SANOFI BIOTECHNOLOGY, Paris (FR); REGENERON PHARMACEUTICALS, INC., Tarrytown, NY (US)

(72) Inventors: Deborah BAUER, Bridgewater, NJ (US); Alexander BODDY, Bridgewater, NJ (US); Neil GRAHAM, Tarrytown, NY (US); Yong LIN, Bridgewater, NJ (US); Janie PARRINO, Tarrytown, NY (US); Rahul PATEL, Bridgewater, NJ (US); Janet VAN ADELSBERG, New York, NY (US); Hubert VAN HOOGSTRATEN, Bridgewater, NJ (US)

(21) Appl. No.: 16/082,841

(22) PCT Filed: Mar. 7, 2017
(86) PCT No.: PCT/US2017/021149

§ 371 Date: Sep. 6, 2018

(30) Foreign Application Priority Data
Mar. 7, 2016 (EP) 16305253.3
May 20, 2016 (EP) 16170664.3
Sep. 5, 2016 (EP) 16306111.2

(51) Int. Cl. C07K 16/24; A61P 19/02; C07K 16/28; A61K 39/395

(52) U.S. Cl. CPC C07K 16/248; A61P 19/02; A61K 2039/545; A61K 39/3955; C07K 16/2866

(57) ABSTRACT

The present invention relates to the use of an anti-IL6 receptor antibody in monotherapy for treating rheumatoid arthritis and for improving the physical function and the quality of life of a subject suffering from rheumatoid arthritis.

### RELATED APPLICATIONS

This application claims priority to European patent application number 16305253.3, filed Mar. 7, 2016; European patent application number 16170664.3, filed May 20, 2016, and European patent application number 16306111.2, filed Sep. 5, 2016, each of which is incorporated herein by reference in its entirety.

### FIELD OF THE INVENTION

The present invention relates to the field of rheumatoid arthritis. More specifically, the invention relates to methods of improving the physical function of subjects suffering from rheumatoid arthritis, methods of improving the health related quality of life of subjects suffering from rheumatoid arthritis, and methods of treating rheumatoid arthritis in subjects suffering from rheumatoid arthritis, comprising administering to the subject an anti-IL6 receptor antibody in monotherapy.

### BACKGROUND

It is estimated that approximately 0.5% to 1% of the adult population in North America and Europe is affected by rheumatoid arthritis (RA). RA affects women twice as often as men and the incidence is highest among women over 40 years of age.

RA is characterized by persistent synovitis and progressive destruction of cartilage and bone in multiple joints. The hallmark of the disease is a symmetric polyarthritis characteristically involving the small joints of the hands and feet. The inflammatory process can also target other organs, characteristically bone marrow (anemia), eye (scleritis, episcleritis), lung (interstitial pneumonitis, pleuritis), cardiac (pericarditis) and skin (nodules, leukocytoclastic vasculitis). Systemic inflammation is characterized by laboratory abnormalities, such as anemia, elevated erythrocyte sedimentation rate, fibrinogen and C-reactive protein (CRP) and by clinical symptoms of fatigue, weight loss, muscle atrophy in affected joint areas. The presence of polyclonal high-titer rheumatoid factors and anticyclic citrullinated peptide (anti-CCP) antibodies provides evidence of immune dysregulation. It has been estimated that 65% to 70% of RA patients have progressive disease that leads to joint destruction, disability and premature death.

In addition to improving the clinical symptoms of RA patients, there is a growing interest in improving the physical function and the health related quality of life of RA patients. Indeed, in addition to the usual instruments intended to assess health status of the patients (presence or absence of disease), physicians and clinicians developed new instruments to measure the physical function and quality of life of the patients, which are useful parameters for the physician to assess the overall response of his or her patient to a specific treatment. Quality of life for instance goes beyond the impairment/disability and handicap continuum by asking what patients' health status prevents them from doing and also about their emotional response to these restrictions. Quality of life also reflects the influences of the personal, social and economic resources that an individual has and the way in which these interact with health status (British Journal of Rheumatology 1997; 36:884-888). The physical function assessment of RA patients typically takes into account the fine movements of the upper extremity, locomotor activities of the lower extremity, and activities that involve both the upper and lower extremities. These parameters are now widely used by the physicians, clinicians and regulatory agencies to compare the different treatment options offered to RA patients. In certain instances, two treatments with a similar efficacy profile may have different quality of life or physical function improvement profiles.

### SUMMARY

The inventors of the present invention have shown that an anti-IL6 receptor antibody, administered as a monotherapy, is capable of showing a remarkable efficacy for treating RA, and is also capable of remarkably improving the physical function and the quality of life of subjects suffering from RA.

Examples of embodiments of the invention are listed below:

### EMBODIMENT 1

A method of improving the physical function of a subject suffering from rheumatoid arthritis, comprising administering to the subject an antibody, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks,
the subject is not administered with any other Disease-Modifying Antirheumatic Drug (DMARD) in course of administration with the antibody, and
the subject was previously ineffectively treated for rheumatoid arthritis by administering at least one DMARD different from the antibody.

### EMBODIMENT 2

The method according to embodiment 1, wherein the subject achieves after at least 24 weeks of administration of the antibody a change from baseline (BL) in the Health Assessment Questionnaire Disability Index (HAQ-DI) of at least 0.22.

### EMBODIMENT 3

The method according to embodiment 1 or 2, wherein the subject achieves after at least 24 weeks of administration of the antibody a change from baseline (BL) in the Health Assessment Questionnaire Disability Index (HAQ-DI) of at least 0.30.

### EMBODIMENT 4

The method according to any one of embodiments 1-3, wherein the subject achieves after at least 24 weeks of administration of the antibody a change from baseline (BL) in the Health Assessment Questionnaire Disability Index (HAQ-DI) of at least 0.40.

### EMBODIMENT 5

The method according to any one of embodiments 1-4, wherein the subject achieves after at least 24 weeks of administration of the antibody a change from baseline (BL) in the Health Assessment Questionnaire Disability Index (HAQ-DI) of at least 0.50.

### EMBODIMENT 6

The method according to any one of embodiments 1-5, wherein the subject achieves after at least 24 weeks of administration of the antibody a change from baseline (BL) in the Health Assessment Questionnaire Disability Index (HAQ-DI) of at least 0.60.

### EMBODIMENT 7

The method according to any one of embodiments 1-6, wherein the subject achieves after at least 24 weeks of administration of the antibody a change from baseline (BL) in the Health Assessment Questionnaire Disability Index (HAQ-DI) of at least 0.61.

### EMBODIMENT 8

A method of improving the health related quality of life of a subject suffering from rheumatoid arthritis, comprising administering to the subject an antibody, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks,
the subject is not administered with any other Disease-Modifying AntiRheumatic Drug (DMARD) in course of administration with the antibody, and
the subject was previously ineffectively treated for rheumatoid arthritis by administering at least one DMARD different from the antibody.

### EMBODIMENT 9

The method according to embodiment 8, wherein the subject achieves after at least 24 weeks of administration of the antibody a change from baseline (BL) in the Short Form-36 Physical Component Summary (SF-36 PCS) of at least 2.5.

### EMBODIMENT 10

The method according to embodiment 8 or 9, wherein the subject achieves after at least 24 weeks of administration of the antibody a change from baseline (BL) in the Short Form-36 Physical Component Summary (SF-36 PCS) of at least 3.

### EMBODIMENT 11

The method according to any one of embodiments 8-10, wherein the subject achieves after at least 24 weeks of administration of the antibody a change from baseline (BL) in the Short Form-36 Physical Component Summary (SF-36 PCS) of at least 4.

### EMBODIMENT 12

The method according to any one of embodiments 8-11, wherein the subject achieves after at least 24 weeks of administration of the antibody a change from baseline (BL) in the Short Form-36 Physical Component Summary (SF-36 PCS) of at least 5.

### EMBODIMENT 13

The method according to any one of embodiments 8-12, wherein the subject achieves after at least 24 weeks of administration of the antibody a change from baseline (BL) in the Short Form-36 Physical Component Summary (SF-36 PCS) of at least 6.

### EMBODIMENT 14

The method according to any one of embodiments 8-13, wherein the subject achieves after at least 24 weeks of administration of the antibody a change from baseline (BL) in the Short Form-36 Physical Component Summary (SF-36 PCS) of at least 7.

### EMBODIMENT 15

The method according to any one of embodiments 8-14, wherein the subject achieves after at least 24 weeks of administration of the antibody a change from baseline (BL) in the Short Form-36 Physical Component Summary (SF-36 PCS) of at least 8.

### EMBODIMENT 16

The method according to any one of embodiments 8-15, wherein the subject achieves after at least 24 weeks of administration of the antibody a change from baseline (BL) in the Short Form-36 Physical Component Summary (SF-36 PCS) of 8.74.

### EMBODIMENT 17

A method of treating rheumatoid arthritis in a subject, comprising administering to the subject an antibody, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks,
the subject is not administered with any other Disease-Modifying AntiRheumatic Drug (DMARD) in course of administration with the antibody, and
the subject was previously ineffectively treated for rheumatoid arthritis by administering at least one DMARD different from the antibody.

### EMBODIMENT 18

The method according to embodiment 17, wherein the subject achieves after at least 24 weeks of administration of the antibody a 20% improvement in the American College of Rheumatology core set disease index (ACR20).

### EMBODIMENT 19

The method according to embodiment 17 or 18, wherein the subject achieves after at least 24 weeks of administration of the antibody a 50% improvement in the American College of Rheumatology core set disease index (ACR50).

### EMBODIMENT 20

The method according to any one of embodiments 17-19, wherein the subject achieves after at least 24 weeks of administration of the antibody a 70% improvement in the American College of Rheumatology core set disease index (ACR70).

### EMBODIMENT 21

The method according to any one of embodiments 17-20, wherein the subject achieves after at least 24 weeks of administration of the antibody a change from baseline (BL) in the Disease Activity Score 28—Erythrocyte Sedimentation Rate (DAS28-ESR) of at least 2.

### EMBODIMENT 22

The method according to any one of embodiments 17-21, wherein the subject achieves after at least 24 weeks of administration of the antibody a change from baseline (BL) in the Disease Activity Score 28—Erythrocyte Sedimentation Rate (DAS28-ESR) of at least 2.5.

### EMBODIMENT 23

The method according to any one of embodiments 17-22, wherein the subject achieves after at least 24 weeks of administration of the antibody a change from baseline (BL) in the Disease Activity Score 28—Erythrocyte Sedimentation Rate (DAS28-ESR) of at least 3, of at least 3.2, or of about 3.28.

### EMBODIMENT 24

The method according to any one of embodiments 17-23, wherein the subject achieves after at least 24 weeks of administration of the antibody a DAS28-ESR score below 3.2.

### EMBODIMENT 25

The method according to any one of embodiments 17-23, wherein the subject achieves after at least 24 weeks of administration of the antibody a DAS28-ESR score below 2.6.

### EMBODIMENT 26

The method according to any one of embodiments 1-25, wherein the subject, who was previously ineffectively treated for rheumatoid arthritis by administering one or more DMARD different from the antibody, is a subject who had an inadequate response or intolerance to one or more Disease-Modifying Anti-Rheumatic Drugs (DMARDs).

### EMBODIMENT 27

The method according to any one of embodiments 1-26, wherein the subject, who was previously ineffectively treated for rheumatoid arthritis by administering at least one DMARD different from the antibody, is a subject who was previously ineffectively treated for rheumatoid arthritis by administering methotrexate.

### EMBODIMENT 28

The method according to any one of embodiments 1-27, wherein the subject who was previously ineffectively treated for rheumatoid arthritis by administering one or more DMARD different from the antibody, is a subject who had an inadequate response or intolerance to methotrexate.

### EMBODIMENT 29

The method according to any one embodiments 1-28, wherein the subject suffering from rheumatoid arthritis is a subject suffering from moderately to severely active rheumatoid arthritis.

### EMBODIMENT 30

The method according to any one of embodiments 1-29, wherein the antibody is administered with a prefilled syringe.

### EMBODIMENT 31

The method according to any one of embodiments 1-30, wherein the antibody is administered with an auto-injector.

### EMBODIMENT 32

The method according to any one of embodiments 1-31, wherein the antibody is administered as an aqueous buffered solution at pH 6.0 containing 21 mM histidine, 45 mM arginine, 0.2% (w/v) polysorbate 20, 5% (w/v) sucrose.

### EMBODIMENT 33

The method according to embodiment 32, wherein the solution comprises at least 130 mg/mL of the antibody.

### EMBODIMENT 34

The method according to embodiment 32, wherein the solution comprises 131.6 mg/mL of the antibody.

### EMBODIMENT 35

The method according to embodiment 32, wherein the solution comprises 175 mg/mL of the antibody.

### EMBODIMENT 36

The method according to any one of embodiments 1-35, wherein the antibody (comprising the heavy chain variable region comprising sequence SEQ ID NO: 1 and the light chain variable region comprising sequence SEQ ID NO: 2) is sarilumab.

### EMBODIMENT 37

An antibody for use in a method of improving the physical function of a subject suffering from rheumatoid arthritis, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks to the subject,
the subject is not administered with any other disease-modifying antirheumatic drug (DMARD) in course of administration with the antibody,
the subject was previously ineffectively treated for rheumatoid arthritis by administering at least one DMARD different from the antibody.

### EMBODIMENT 38

The antibody for use according to embodiment 37, wherein the subject achieves after at least 24 weeks of administration of the antibody a change from baseline (BL) in the Health Assessment Questionnaire Disability Index (HAQ-DI) of at least 0.22.

### EMBODIMENT 39

The antibody for use according to embodiment 37 or 38, wherein the subject achieves after at least 24 weeks of administration of the antibody a change from baseline (BL) in the Health Assessment Questionnaire Disability Index (HAQ-DI) of at least 0.30.

### EMBODIMENT 40

The antibody for use according to any one of embodiments 37-39, wherein the subject achieves after at least 24 weeks of administration of the antibody a change from baseline (BL) in the Health Assessment Questionnaire Disability Index (HAQ-DI) of at least 0.40.

### EMBODIMENT 41

The antibody for use according to any one of embodiments 37-40, wherein the subject achieves after at least 24 weeks of administration of the antibody a change from baseline (BL) in the Health Assessment Questionnaire Disability Index (HAQ-DI) of at least 0.50.

### EMBODIMENT 42

The antibody for use according to any one of embodiments 37-41, wherein the subject achieves after at least 24 weeks of administration of the antibody a change from baseline (BL) in the Health Assessment Questionnaire Disability Index (HAQ-DI) of at least 0.60.

### EMBODIMENT 43

The antibody for use according to any one of embodiments 37-42, wherein the subject achieves after at least 24 weeks of administration of the antibody a change from baseline (BL) in the Health Assessment Questionnaire Disability Index (HAQ-DI) of at least 0.61.

### EMBODIMENT 44

An antibody for use in a method of improving the health related quality of life of a subject suffering from rheumatoid arthritis, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks to the subject,
the subject is not administered with any other disease-modifying antiRheumatic drug (DMARD) in course of administration with the antibody, and
the subject was previously ineffectively treated for rheumatoid arthritis by administering at least one DMARD different from the antibody.

### EMBODIMENT 45

The antibody for use according to embodiment 44, wherein the subject achieves after at least 24 weeks of administration of the antibody a change from baseline (BL) in the Short Form-36 Physical Component Summary (SF-36 PCS) of at least 2.5.

### EMBODIMENT 46

The antibody for use according to embodiment 44 or 45, wherein the subject achieves after at least 24 weeks of administration of the antibody a change from baseline (BL) in the Short Form-36 Physical Component Summary (SF-36 PCS) of at least 3.

### EMBODIMENT 47

The antibody for use according to any one of embodiments 44-46, wherein the subject achieves after at least 24 weeks of administration of the antibody a change from baseline (BL) in the Short Form-36 Physical Component Summary (SF-36 PCS) of at least 4.

### EMBODIMENT 48

The antibody for use according to any one of embodiments 44-47, wherein the subject achieves after at least 24 weeks of administration of the antibody a change from baseline (BL) in the Short Form-36 Physical Component Summary (SF-36 PCS) of at least 5.

### EMBODIMENT 49

The antibody for use according to any one of embodiments 44-48, wherein the subject achieves after at least 24 weeks of administration of the antibody a change from baseline (BL) in the Short Form-36 Physical Component Summary (SF-36 PCS) of at least 6.

### EMBODIMENT 50

The antibody for use according to any one of embodiments 44-49, wherein the subject achieves after at least 24 weeks of administration of the antibody a change from baseline (BL) in the Short Form-36 Physical Component Summary (SF-36 PCS) of at least 7.

### EMBODIMENT 51

The antibody for use according to any one of embodiments 44-50, wherein the subject achieves after at least 24 weeks of administration of the antibody a change from baseline (BL) in the Short Form-36 Physical Component Summary (SF-36 PCS) of at least 8.

### EMBODIMENT 52

The antibody for use according to any one of embodiments 44-51, wherein the subject achieves after at least 24 weeks of administration of the antibody a change from baseline (BL) in the Short Form-36 Physical Component Summary (SF-36 PCS) of 8.74.

### EMBODIMENT 53

An antibody for use in a method of treating rheumatoid arthritis in a subject, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks to the subject,
the subject is not administered with any other disease-modifying antiRheumatic drug (DMARD in course of administration with the antibody, and
the subject was previously ineffectively treated for rheumatoid arthritis by administering at least one DMARD different from the antibody.

### EMBODIMENT 54

The antibody for use according to embodiment 53, wherein the subject achieves after at least 24 weeks of administration of the antibody a 20% improvement in the American College of Rheumatology core set disease index (ACR20).

### EMBODIMENT 55

The antibody for use according to embodiment 53 or 54, wherein the subject achieves after at least 24 weeks of administration of the antibody a 50% improvement in the American College of Rheumatology core set disease index (ACR50.

### EMBODIMENT 56

The antibody for use according to any one of embodiments 53-55, wherein the subject achieves after at least 24 weeks of administration of the antibody a 70% improvement in the American College of Rheumatology core set disease index (ACR70).

### EMBODIMENT 57

The antibody for use according to any one of embodiments 53-56, wherein the subject achieves after at least 24 weeks of administration of the antibody a change from baseline (BL) in the Disease Activity Score 28—Erythrocyte Sedimentation Rate (DAS28-ESR) of at least 2.

### EMBODIMENT 58

The antibody for use according to any one of embodiments 53-57, wherein the subject achieves after at least 24 weeks of administration of the antibody a change from baseline (BL) in the Disease Activity Score 28—Erythrocyte Sedimentation Rate (DAS28-ESR) of at least 2.5.

### EMBODIMENT 59

The antibody for use according to any one of embodiments 53-58, wherein the subject achieves after at least 24 weeks of administration of the antibody a change from baseline (BL) in the Disease Activity Score 28—Erythrocyte Sedimentation Rate (DAS28-ESR) of at least 3, of at least 3.2, or of about 3.28.

### EMBODIMENT 60

The antibody for use according to any one of embodiments 53-59, wherein the subject achieves after at least 24 weeks of administration of the antibody a DAS28-ESR score below 3.2.

### EMBODIMENT 61

The antibody for use according to any one of embodiments 53-60, wherein the subject achieves after at least 24 weeks of administration of the antibody a DAS28-ESR score below 2.6.

### EMBODIMENT 62

The antibody for use according to any one of embodiments 37-61, wherein the subject, who was previously ineffectively treated for rheumatoid arthritis by administering one or more DMARD different from the antibody, is a subject who had an inadequate response or intolerance to one or more disease-modifying anti-rheumatic drugs (DMARDs).

### EMBODIMENT 63

The antibody for use according to any one of embodiments 37-62, wherein the subject, who was previously ineffectively treated for rheumatoid arthritis by administering at least one DMARD different from the antibody, is a subject who was previously ineffectively treated for rheumatoid arthritis by administering methotrexate.

### EMBODIMENT 64

The antibody for use according to any one of embodiments 37-63, wherein the subject, who was previously ineffectively treated for rheumatoid arthritis by administering one or more DMARD different from the antibody, is a subject who had an inadequate response or intolerance to methotrexate.

### EMBODIMENT 65

The antibody for use according to any one embodiments 37-64, wherein the subject suffering from rheumatoid arthritis is a subject suffering from moderately active rheumatoid to severely active rheumatoid arthritis.

### EMBODIMENT 66

The antibody for use according to any one of embodiments 37-65, wherein the antibody is administered with a prefilled syringe.

### EMBODIMENT 67

The antibody for use according to any one of embodiments 37-66, wherein the antibody is administered with an auto-injector.

### EMBODIMENT 68

The antibody for use according to any one of embodiments 37-67, wherein the antibody is administered as an aqueous buffered solution at pH 6.0 containing 21 mM histidine, 45 mM arginine, 0.2% (w/v) polysorbate 20, 5% (w/v) sucrose.

### EMBODIMENT 69

The antibody for use according to embodiment 68, wherein the solution comprises at least 130 mg/mL of the antibody.

### EMBODIMENT 70

The antibody for use according to embodiment 68, wherein the solution comprises 131.6 mg/mL of the antibody.

### EMBODIMENT 71

The antibody for use according to embodiment 68, wherein the solution comprises 175 mg/mL of the antibody

### EMBODIMENT 72

The antibody for use according to any one of embodiments 37-71, wherein the antibody (comprising the heavy chain variable region comprising sequence SEQ ID NO: 1 and the light chain variable region comprising sequence SEQ ID NO: 2) is sarilumab.

### EMBODIMENT 73

The antibody for use according to any one of embodiments 37-72, wherein the antibody is administered subcutaneously at 150 mg once every two weeks to the subject.

### EMBODIMENT 74

The antibody for use according to any one of embodiments 37-72, wherein the antibody is administered subcutaneously at 200 mg once every two weeks to the subject.

### EMBODIMENT 75

The antibody for use according to any of the previous embodiments, wherein the subject is intolerant of one or more DMARDs.

### EMBODIMENT 76

The antibody for use according to embodiment 75, wherein the DMARD is methotrexate.

### EMBODIMENT 77

The antibody for use according to any of the previous embodiments, wherein the subject has moderately to severely active rheumatoid arthritis and has had an inadequate response or has an intolerance to one or more disease-modifying antirheumatic drugs.

### EMBODIMENT 78

The antibody for use according to embodiment 77, wherein the DMARD is methotrexate.

### EMBODIMENT 79

The antibody for use according to any of the previous embodiments, wherein the subject suffers from diminishment in quality of life due to RA.

### EMBODIMENT 80

The antibody for use according to embodiment 77, wherein the subject scores as more severe than average on a metric selected from Change From Baseline in European Quality of Life-5 Dimension 3 Level (EQ-5D-3L), Change From Baseline in Rheumatoid Arthritis Impact of Disease (RAID), Work Days Missed Due to Arthritis, Work Productivity Reduced by 50% Due to Arthritis, Rate of Arthritis Interference With Work Productivity, House Work Days Missed Due to Arthritis, Days With Household Work Productivity Reduced by 50% Due to Arthritis, Days With Family/Social/Leisure Activities Missed Due to Arthritis, Days With Outside Help Hired Due to Arthritis, Rate of RA Interference With Household Work Productivity, Morning Stiffness VAS, Individual ACR Component—TJC and SJC, Individual ACR Component—Physician Global VAS, Participant Global VAS and Pain VAS, and Individual ACR Component—ESR Level.

### EMBODIMENT 81

The antibody for use according to any of the previous embodiments, wherein the antibody is administered subcutaneously at 150 mg once every two weeks to the subject.

### EMBODIMENT 82

The antibody for use according to any of embodiments 37-80, wherein the antibody is administered subcutaneously at 200 mg once every two weeks to the subject.

### EMBODIMENT 83

A composition comprising the antibody of any of embodiments 37-82.

### EMBODIMENT 84

The use of an antibody for the manufacture of a medicament for improving the physical function of a subject suffering from rheumatoid arthritis, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks,
the subject is not administered with any other DMARD in course of administration with the antibody, and
the subject was previously ineffectively treated for rheumatoid arthritis by administering at least one DMARD different from the antibody.

### EMBODIMENT 85

The use of an antibody for the manufacture of a medicament for improving the health related quality of life of a subject suffering from rheumatoid arthritis, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks,
the subject is not administered with any other DMARD in course of administration with the antibody, and
the subject was previously ineffectively treated for rheumatoid arthritis by administering at least one DMARD different from the antibody.

### EMBODIMENT 86

The use of an antibody for the manufacture of a medicament for treating rheumatoid arthritis in a subject, wherein:
the antibody comprises the heavy chain variable region comprising sequence SEQ ID NO:1 and the light chain variable region comprising sequence SEQ ID NO:2,
the antibody is administered subcutaneously at 150 mg or 200 mg once every two weeks,
the subject is not administered with any other DMARD in course of administration with the antibody, and the subject was previously ineffectively treated for rheumatoid arthritis by administering at least one DMARD different from the antibody.

### DETAILED DESCRIPTION

It has been shown by the inventors that a anti-IL6R antibody administered as a monotherapy, is efficient for treating rheumatoid arthritis. In addition, the inventors have shown that the antibody administered as a monotherapy is also effective for improving the physical function and the Quality of Life of subjects suffering from rheumatoid arthritis.

According to the invention, “monotherapy” means that the subject receiving the antibody is not administered with any other DMARD in course of administration with the antibody.

The efficacy of the antibody for treating rheumatoid arthritis is typically measured using the standard methods in the field, commonly used by the clinicians and the rheumatologists, for example the DAS-28 and ACR parameters, for example the DAS-28 ESR, ACR20, ACR50 and ACR70 parameters.

The improvement of the physical function of the subjects suffering from rheumatoid arthritis is in various embodiments measured using the standard methods in the field, commonly used by the clinicians and the rheumatologists, namely the HAQ-DI parameter.

The improvement of the quality of life of the subjects suffering from rheumatoid arthritis is typically measured using the standard methods in the field, commonly used by the clinicians and the rheumatologists, for example the SF36 parameter, and in some embodiments the SF-36 PCS parameter.

The baseline (also referred to herein as “BL”) is defined as the score obtained by the subject before being administered with the antibody according to the invention.

Change from baseline is defined as the difference existing between the score obtained by the subject at baseline and the score obtained by the subject after being administered with the antibody according to the invention, for example measured at least 24 weeks after the first administration of the antibody, including 24 weeks after the first administration of the antibody.

### DAS28-ESR

DAS28 is a composite score that includes 4 variables:
Tender Joints Count (based on 28 joints: shoulder (n=2), elbow (n=2), wrist (n=2), metacarpophalageal (n=10), interphalangeal of thumb (n=2), proximal interphalangeal (n=8), knee (n=2))
Swollen Joints count (based on 28 joints: shoulder (n=2), elbow (n=2), wrist (n=2), metacarpophalageal (n=10), interphalangeal of thumb (n=2), proximal interphalangeal (n=8), knee (n=2))
General health assessment (GH) by the patient assessed from the ACR RA core set questionnaire (patient global assessment) in 100 mm visual analogue scale (VAS)
Marker of inflammation assessed by the CRP in mg/L or ESR in mm/hr.
   It is a continuous measure allowing for measurement of absolute change in disease activity and percentage improvement. The DAS28-ESR can be calculated using the following formula:

DAS28-ESR=0.56×\sqrt{28TJC}+0.28×28SJC+0.70×Ln[ESR(mm/h)]+0.014×GH(VAS)

The DAS28-ESR score provides a number indicating the current disease activity of the RA. A DAS28-ESR score above 5.1 means high disease activity, whereas a DAS28-ESR score below 3.2 indicates low disease activity and a DAS28-ESR score below 2.6 means disease remission.
When calculating the 28TJC and 28SJC, with individual missing joint scores (the ‘replaced or fused’ joints are not taken into consideration for the swelling or tenderness) imputed as the mean of the scored joints, the tender/swollen joint counts after imputation are as follows:
28TJC/28SJC=sum (scored tender/swollen joints)*(number of joints in the full joint set/number of scored tender/swollen joints). The number of joints in the full joint set is defined as (28—number of replaced or fused joints) and the scored joints refer to those with an answer (0—no pain, 1—pain).
If the subject answers that he or she is experiencing no pain (i.e., ESR=0), then for purposes of calculating the DAS-ESR score one should instead insert the value ESR=1 for the purpose of the DAS28-ESR calculation to enable a non-missing score. DAS28-ESR is considered as missing if one of the components is missing.

### Visual Analog Score (VAS)

The VAS is a measure to assess patient-related rheumatoid arthritis severity. Patient makes a vertical mark through each of two lines which best describes the amount of pain due to rheumatoid arthritis. Range from no pain to most severe pain.

### ACR20

To be classified as an ACR20 responder, a patient must achieve 20% improvement compared with baseline, in both TJC and SJC, as well as 20% improvement in at least 3 out of the 5 remaining ACR components: physician's global assessment of disease activity, patient's global assessment of disease activity, pain, HAQ-DI, and CRP.

### ACR50

ACR50 is defined as the event of achieving at least 50% improvement in both TJC and SJC, and at least 50% improvement in at least 3 out of the 5 remaining ACR components.

### ACR70

ACR70 is defined as the event of achieving at least 70% improvement in both TJC and SJC and at least 70% improvement in at least 3 out of the 5 remaining ACR components.

The 7 ACR components assessing the signs and symptoms of RA are defined below (A-G):

### A) Tender Joint Count (TJC)

A total of 68 joints are assessed for tenderness. The 68 joints to be examined for tenderness are: temporomandibular (n=2), sternoclavicular (n=2), acromioclavicular (n=2), shoulder (n=2), elbow (n=2), wrist (n=2), metacarpophalageal (n=10), interphalangeal of thumb (n=2), distal interphalangeal (n=8), proximal interphalangeal (n=8), hip (n=2), knee (n=2), ankle mortise (n=2), ankle tarsus (n=2), metatarsophalangeal (n=10), interphalangeal of great toe (n=2), and proximal/distal interphalangeal of the toes (n=8).

A formal count of the joints is performed by a trained assessor. Joint tenderness is defined as pain induced by the pressure of the joints, exerted by the assessor's thumb and index finger. The assessor classifies each joint as painful (yes/no) and swollen (yes/no). A score of 0/1 is given to each tender joint with 0 representing no pain and 1 representing pain. The tender joint count ranges from 0 to 68 where 0 is considered the best and 68 the worst.

### B) Swollen Joint Count (SJC)

The 66 joints to be examined for swelling are the same as those examined for tenderness, except the hip joints are not included. A formal count of the joints is performed by a trained assessor. The assessor classifies each joint as swollen (yes/no). A score of 0/1 is given to each swollen joint with 0 representing no swollen and 1 representing a swollen joint. The swollen joint count ranges from 0 to 66 where 0 is considered the best and 66 the worst.

### C) Physician's Global Assessments of Disease Activity

Physician's global assessments of the patient's current disease activity is assessed on an anchored 100 mm horizontal VAS where 0 is considered the best disease activity (no disease activity) and 100 the worst (most disease activity).

### D) Patient's Global Assessments of Disease Activity

Patient's global assessments of their current disease activity is rated on an anchored 100 mm horizontal VAS where 0 is considered the best disease activity (no disease activity) and 100 the worst (most disease activity).

### E) Patient's Assessment of Pain

Patients are requested to indicate their pain intensity due to their RA using a 100 mm horizontal VAS where 0 is considered “No pain” and 100 “the worst pain you can imagine”.

F) Patient's Assessment of Physical function—Health Assessment Questionnaire Disease Index (HAQ-DI)

The HAQ-DI is a standardized questionnaire developed for use in RA. The HAQ-DI, with the past week as the time frame, focuses on whether the respondent “is able to . . . ” do the activity and covers 8 categories in 20 items: dressing and grooming, arising, eating, walking, hygiene, reach, grip and activities, for which there are at least 2 questions by category. The 4 responses for the HAQ-DI questions are graded as follows: without any difficulty=0; with some difficulty=1; with much difficulty=2; and unable to do=3. To calculate the Standard HAQ-DI Score (With Aids/Devices), there are 3 steps:

1. Sum the 8 category scores by using the highest sub-category score from each category. For example, in the category EATING there are 3 sub-category items. A patient responds with a 1, 2, and 0, respectively; the category score is 2.
2. Adjust for use of aids/devices and/or help from another person when indicated.
Adjust the score for a category by increasing a 0 or a 1 to a 2.
If a patient's highest score for that sub-category is a 2 it remains a 2, and if a 3, it remains a 3.
The data entered at field “Other specify” are not used for score adjustment.
   3. Divide the summed category scores by the number of categories answered (must be a minimum of 6) to obtain a HAQ-DI score of 0 to 3 (3=worst functioning).

A HAQ-DI score cannot be calculated validly when there are scores for less than 6 of the 8 categories. HAQ-DI scoring ranges between 0 and 3. A high HAQ-DI score has been found to be a strong predictor of morbidity and mortality in RA. A 0.22 unit difference is considered clinically meaningful.

### G) The Level of an Acute Phase Reactant Measured by CRP

High sensitivity CRP is assessed centrally. Since CRP levels are directly correlated with Interleukin 6 (IL-6) receptor activity, it is expected that active dose regimens has a dramatic lowering effect on CRP levels. Therefore, during the study, post-dosing CRP remains blinded to the investigators, the sponsor and the patients. The ACR components are further described in Table 1.

TABLE 1
ACR components

| ACR components | Range | Direction |
|---|---|---|
| TJC | 0 to 68 | Lower is better |
| SJC | 0 to 66 | Lower is better |
| Pain VAS | 0 to 100 | Lower is better |
| Patient global VAS | 0 to 100 | Lower is better |
| Physician global VAS | 0 to 100 | Lower is better |
| HAQ-DI | 0 to 3 | Lower is better |
| CRP (mg/dL) | >0 | Lower is better |

TJC: Total Joint Counts,
SJC: Swollen Joint Counts,
VAS: Visual Analog Scale

### SF-36 V2

The QualityMetric's SF-36v2® Health Survey is a multi-purpose, short-form health survey with 36 questions. It yields scores for eight domains (Physical Functioning, Role-Physical, Bodily pain, General health, Vitality, Social Functioning, Role-Emotional, and Mental Health, where each domain is scored from 0 to 100 and where higher scores indicate better health and well-being), as well as two summary measures of physical and mental health: the physical component summary (PCS) and mental component summary (MCS).

The scoring process is summarized below:
1. Enter item response data.
2. Recode item response values.
3. Determine health domain scale raw scores.
4. Transform health domain scale raw scores to 0 to 100 scores.
5. Transform health domain scale 0 to 100 to normal-based scores.
6. Score PCS and MCS measures.
The following table shows the construction and summary measures of the SF-36 scales:

TABLE
SF-36 V2 measurement model

| Summary | | |
|---|---|---|
| Measures | Scales | Items |
| PCS* | Physical Functioning (PF) | 3a, 3b, 3c, 3d, 3e, 3f, 3g, 3h, |
| | | 3i, 3j |
| | Role-Physical (RP) | 4a, 4b, 4c, 4d |
| | Bodily Pain (BP) | 7, 8 |
| | General Health (GH) | 1, 11a, 11b, 11c, 11d |
| MCS) | Vitality (VT) | 9a, 9e, 9g, 9i |
| | Social Functioning (SF) | 6, 10 |
| | Role-Emotional (RE) | 5a, 5b, 5c |
| | Mental Health (MH) | 9b, 9c, 9d, 9f, 9h |

*MCS and PCS derived from the eight scales (Ware, J E. et al. 1994 “SF-36 Physical and Mental Health Summary Scales: A Users' Manual”. Boston: The Health Institute)

The Abbreviated Item Content is as follows:
3a Vigorous activities, such as running, lifting heavy objects, or participating in strenuous sports;
3b Moderate activities, such as moving a table, pushing a vacuum cleaner, bowling, or playing golf;
3c Lifting or carrying groceries;
3d Climbing several flights of stairs;

3e Climbing one flight of stairs;

3f Bending, kneeling, or stooping;
3g Walking more than a mile;
3h Walking several hundred yards;
3i Walking one hundred yards;
3j Bathing or dressing oneself;
4a Cut down the amount of time one spent on work or other activities;
4b Accomplished less than you would like;
4c Limited in kind of work or other activities;
4d Had difficulty performing work or other activities (e.g., it took extra effort);
7 Intensity of bodily pain;
8 Extent pain interfered with normal work;
1 Is your health: excellent, very good, good, fair, poor;
11a Seem to get sick a little easier than other people;
11 b As healthy as anybody I know;
11c Expect my health to get worse;
11d Health is excellent;
9a Feel full of life;
9e Have a lot of energy;
9g. Feel worn out;
9i Feel tired;
6 Extent health problems interfered with normal social activities;
10 Frequency health problems interfered with social activities;
5a Cut down the amount of time spent on work or other activities;
5b Accomplished less than you would like;
5c Did work or other activities less carefully than usual;
9b Been very nervous;
9c Felt so down in the dumps that nothing could cheer you up;
9d Felt calm and peaceful;
9f Felt downhearted and depressed; and
9h Been happy.

The PCS and MCS summary measure scores are computed if at least 50% of the component scales are available. The scale scores are computed if at least 50% of items are available within the corresponding scale. The missing items are imputed by the mean of available items.

### General Scoring Information

Items and scales are scored in 3 steps:
Step 1. Item recoding, for the 10 items that require recoding,
Step 2. Computing scale scores by summing across items in the same scale (raw scale scores); and,
Step 3. Transforming raw scale scores to a 0-100 scale (transformed scale).

### Item Recoding

All 36 items should be checked for out-of-range values prior to assigning the final item value. All out-of-range values should be recoded as missing data.

The following tables show the recoding of response choice.

### How to Treat Missing Data

A scale score is calculated if a respondent answered at least half of the items in a multi-item scale (or half plus one in the case of scales with an odd number of items).

The recommended algorithm substitutes a person-specific estimate for any missing item when the respondent answered at least 50 percent of the items in a scale. A psychometrically sound estimate is the average score, across completed items in the same scale, for that respondent. For example, if a respondent leaves one item in the 5-item mental Health scale blank, one must substitute the respondent's average score (across the 4 completed mental health items) for that one item. When estimating the respondent's average score, use the respondent's final item values.

### Computing Raw Scale Scores

After item recoding, including handling of missing data, a raw score is computed for each scale. This score is a simple algebraic sum of responses for all items in that scale.

If the respondent answered at least 50% of the items in a multi-items scale, the score can be calculated. If the respondent did not answer at least 50% of the items, the score for that scale should be set to missing.

### Transformation of the Scale Scores

The next step involves transforming each raw score to a 0 to 100 scale using the following formula:

Transformed scale=[(actual raw score−lowest possible raw score)/possible raw score range]×100

This transformation converts the lowest and highest possible scores to zero and 100, respectively.

TABLE
SF-36 V2 raw scores of eight domains

| | Lowest and Highest | raw score |
|---|---|---|
| Scale | possible raw scores | range |
| Physical Functioning | 10 and30 | 20 |
| Role-Physical | 4 and20 | 16 |
| Bodily pain | 2 and12 | 10 |
| General health | 5 and25 | 20 |
| Vitality | 4 and20 | 16 |
| Social Functioning | 2 and10 | 8 |
| Role-Emotional | 3 and15 | 12 |
| Mental Health | 5 and25 | 20 |

The score of each of the 36 items is collected in CRF (case report form). Then, a SAS (Statistical Analysis System) code (e.g. the one provided by the QualityMetric survey) is used to calculate the eight scales, the two summary measure scores and the standardized summary scores.

The PCS and MCS summary measure scores are computed if at least 50% of the component scales are available. The scale scores are computed if at least 50% of items are available within the corresponding scale. The missing items are imputed by the mean of available items.

Change from BL in SF-36 scores (physical component summary score and mental component summary score as well as the eight domains) is then analyzed.

### SF-36 V2 Scoring

### General Scoring Information:

Items and scales are scored in 3 steps (as instructed by the QualityMetric survey manual):
Step 1. Item recoding, for then 10 items that require recoding
Step 2. Computing scale scores by summing across items in the same scale (raw scale scores); and
Step 3. Transforming raw scale scores to a 0-100 scale (transformed scale)
Step 5: Compute Z-Scores
Step 6: Convert Z-score to Norm Based scores for domains
PCS: Compute aggregate PCS score using a specific weighted formula, convert this into a Norm based score
MCS: Compute aggregate MCS score using a specific weighted formula, convert this into a Norm based score

### Item Recoding:

All 36 items should be checked for out-of-range values prior to assigning the final item value. All out-of-range values should be recoded as missing data.

### How to Treat Missing Data

A scale score is calculated if a respondent answered at least half of the items in a multi-item scale (or half plus one in the case of scales with an odd number of items).

The recommended algorithm substitutes a person-specific estimate for any missing item when the respondent answered at least 50 percent of the items in a scale. A psychometrically sound estimate is the average score, across completed items in the same scale, for that respondent. For example, if a respondent leaves one item in the 5-item mental Health scale blank, one must substitute the respondent's average score (across the 4 completed mental health items) for that one item. When estimating the respondent's average score, use the respondent's final item values.

### Computing Raw Scale Scores

After item recoding, including handling of missing data, a raw score is computed for each scale. This score is a simple algebraic sum of responses for all items in that scale.

If the respondent answered at least 50% of the items in a multi-items scale, the score can be calculated. If the respondent did not answer at least 50% of the items, the score for that scale should be set to missing.

### Transformation of the Scale Scores

The next step involves transforming each raw score to a 0 to 100 scale using the following formula:

Transformed scale=[(actual raw score−lowest possible raw score)/possible raw score range]×100

This transformation converts the lowest and highest possible scores to zero and 100, respectively.

### The Antibody

The present disclosure includes methods that comprise administering to a patient an antibody, or an antigen-binding fragment thereof, that binds specifically to hIL-6R. As used herein, the term “hIL-6R” means a human cytokine receptor that specifically binds human interleukin-6 (IL-6). In certain embodiments, the antibody that is administered to the patient binds specifically to the extracellular domain of hIL-6R.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may in various embodiments be adapted for use in the context of an antigen-binding fragment of an anti-IL-6R antibody using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies featured in the invention may in various embodiments nonetheless include amino acid residues not encoded by human germline immunoglobulin sequences (e.g., mutations introduced by random or site-specific mutagenesis in vitro or by somatic mutation in vivo), for example in the CDRs and in in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-6R, as used herein, includes antibodies that bind IL-6R or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. An isolated antibody that specifically binds human IL-6R may, however, have cross-reactivity to other antigens, such as IL-6R molecules from other (non-human) species.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “KD”, as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IL-6R antibodies useful for the methods featured herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-IL-6R antibodies comprising variants of any of the HCVR, LCVR, and/or CDR amino acid sequences disclosed herein having one or more conservative substitutions. For example, the present invention includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present invention, the anti-IL-6R antibody, or antigen-binding fragment thereof, in various embodiments comprises a heavy chain variable region (HCVR), light chain variable region (LCVR), and/or complementarity determining regions (CDRs) comprising any of the amino acid sequences of the anti-IL-6R antibodies as claimed in U.S. Pat. No. 7,582,298, incorporated herein by reference in its entirety. The anti-IL-6R antibody or antigen-binding fragment thereof that can be used in the context of the methods of the present invention comprises the heavy chain complementarity determining regions (HCDRs) of a HCVR comprising the amino acid sequence of SEQ ID NO:1 and the light chain complementarity determining regions (LCDRs) of a LCVR comprising the amino acid sequence of SEQ ID NO:2. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO:3; the HCDR2 comprises the amino acid sequence of SEQ ID NO:4; the HCDR3 comprises the amino acid sequence of SEQ ID NO:5; the LCDR1 comprises the amino acid sequence of SEQ ID NO:6; the LCDR2 comprises the amino acid sequence of SEQ ID NO:7; and the LCDR3 comprises the amino acid sequence of SEQ ID NO:8. In yet other embodiments, the anti-IL-6R antibody or antigen-binding fragment thereof comprises an HCVR comprising SEQ ID NO:1 and an LCVR comprising SEQ ID NO:2.

In another embodiments, the anti-IL-6R antibody or antigen-binding fragment thereof comprises a heavy chain comprising SEQ ID NO:9 and an light chain comprising SEQ ID NO:10. According to certain exemplary embodiments, the methods of the present invention comprise the use of the anti-IL-6R antibody referred to and known in the art as sarilumab, or a bioequivalent thereof.

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IL-6R antibody are bioequivalent if they are pharmaceutically equivalent, meaning they contain the same amount of active ingredient (e.g., IL-6R antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The invention in certain embodiments relates to methods comprising administering to the subject an antibody which comprises the heavy chain variable region comprising sequence SEQ ID NO:1 and the light chain variable region comprising sequence SEQ ID NO:2.

The disclosure provides pharmaceutical compositions comprising such antibody, and methods of using these compositions.

The antibody which comprises the heavy chain variable region comprising sequence SEQ ID NO:1 and the light chain variable region comprising sequence SEQ ID NO:2 is an antibody that specifically binds human interleukin-6 receptor (hIL-6R). See international publication number WO2007/143168, incorporated herein by reference in its entirety.

In one embodiment, the antibody which comprises the heavy chain variable region comprising sequence SEQ ID NO:1 and the light chain variable region comprising sequence SEQ ID NO:2 is sarilumab.

### DMARDs

DMARDs are drugs defined by their use in rheumatoid arthritis to slow down disease progression.

DMARDs have been classified as synthetic (sDMARD) and biological (bDMARD). Synthetic DMARDs include non-exhaustively methotrexate, sulfasalazine, leflunomide, and hydroxychloroquine. Biological DMARDs include non-exhaustively adalimumab, golimumab, etanercept, abatacept, infliximab, rituximab, and tocilizumab.

### Methods of Administration and Formulations

The antibody in various embodiments is administered to the subject. In various embodiments, the antibody is administered at about 100 mg, 150 mg or about 200 mg once every two weeks. “Once every two weeks” has the same meaning as “q2w” or “once per two weeks”, i.e. that the antibody is administered once in a two week period of time. According to certain embodiments, the antibody is administered subcutaneously.

In certain embodiments, the antibody is administered at about 100 mg, 150 mg or about 200 mg once every two weeks. In this context, “about” refers to an amount within 5% of the stated amount. For example, “about 100 mg” is a range of between 95 and 105 mg. According to certain embodiments, the antibody is administered subcutaneously.

The antibody is administered to the subject in various embodiments in a formulation comprising suitable carriers, excipients, and other agents to provide improved transfer, delivery, tolerance, and the like, and suitable for a subcutaneous injection.

The injectable preparations may be prepared by methods publicly known. For example, injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 20 or 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable preparation thus prepared can be filled in an appropriate ampoule.

The antibody is typically formulated as described herein and in international publication number WO2011/085158, incorporated herein by reference in its entirety.

In various embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose, and
between about 100 mg/mL and about 200 mg/mL of the antibody.

In another embodiment, the antibody is administered as an aqueous buffered solution at pH 6.0 containing
about 21 mM histidine,
about 45 mM arginine,
about 0.2% (w/v) polysorbate 20,
about 5% (w/v) sucrose, and
at least about 130 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose, and
about 131.6 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose; and
about 175 mg/mL of the antibody.

In other embodiments, the antibody is administered as an aqueous buffered solution at pH 6.0 containing
21 mM histidine,
45 mM arginine,
0.2% (w/v) polysorbate 20,
5% (w/v) sucrose, and
between 100 mg/mL and 200 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
at least 130 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
131.6 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose; and
175 mg/mL of the antibody.

The antibody according to the invention can be administered to the subject using any acceptable device or mechanism. For example, the administration can be accomplished using a syringe and needle or with a reusable pen and/or auto-injector delivery device. The methods of the present invention include the use of numerous reusable pen and/or autoinjector delivery devices to administer an antibody (or pharmaceutical formulation comprising the antibody). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to, the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), the HUMIRA™ Pen (Abbott Labs, Abbott Park, Ill.), the DAI® Auto Injector (SHL Group) and any auto-injector featuring the PUSHCLICK™ technology (SHL Group), to name only a few.

In one embodiment, the antibody is administered with a prefilled syringe.

In another embodiment, the antibody is administered with a prefilled syringe containing a safety system. For example, the safety system prevents an accidental needstick injury. In various embodiments, the antibody is administered with a prefilled syringe containing an ÈRIS™ safety system (West Pharmaceutical Services Inc.). See also U.S. Pat. Nos. 5,215,534 and 9,248,242, incorporated herein by reference in their entireties.

In another embodiment, the antibody is administered with an auto-injector. In various embodiments, the antibody is administered with an auto-injector featuring the PUSHCLICK™ technology (SHL Group). In various embodiments, the auto-injector is a device comprising a syringe that allows for administration of a dose of the composition and/or antibody to a subject. See also U.S. Pat. Nos. 9,427,531 and 9,566,395, incorporated herein by reference in their entireties.

### Patient Population

According to the invention, “subject” means a human subject or human patient.

The antibody according to the invention is in various embodiments administered to subjects previously ineffectively treated for rheumatoid arthritis by administering one or more DMARD different from the antibody.

According to the invention, a subject who is considered “ineffectively treated” by his or her physician is a subject who in various embodiments either has shown to be intolerant to the one or more DMARD tested by the physician, and/or a subject who has shown an inadequate response to the one or more DMARD tested by the physician, typically a subject who is still considered by the physician to present with, or to have, active rheumatoid arthritis despite the previous one or more DMARD administered. The “Active rheumatoid arthritis” is typically defined as:
at least 6 of 66 swollen joints and 8 of 68 tender joints, as counted by the physician in a typical quantitative swollen and tender joint count examination,
High sensitivity C-reactive protein (hs-CRP)≥8 mg/L or ESR≥28 mm/H
DAS28ESR>5.1.

In one embodiment, the subject, who was previously ineffectively treated for rheumatoid arthritis by administering at least one DMARD different from the antibody, is a subject who was previously ineffectively treated for rheumatoid arthritis by administering a DMARD. In various embodiments, the DMARD is selected from the group consisting of methotrexate, sulfasalazine, leflunomide, and hydroxychloroquine. In various embodiments, the DMARD is methotrexate.

In another embodiment, the subject, who was previously ineffectively treated for rheumatoid arthritis by administering one or more DMARD different from the antibody, is a subject who had an inadequate response or intolerance to methotrexate.

According to the invention, for those subjects previously ineffectively treated for rheumatoid arthritis by administering one or more DMARD different from the antibody, the one or more DMARD is/are not administered anymore to the subject, and the antibody is in various embodiments administered alone, in monotherapy to the subject.

In various embodiments, the subject is intolerant to the DMARD due to one or more physical reactions, conditions or symptoms from the treatment with the DMARD. Physical reactions, conditions or symptoms can include allergies, pain, nausea, diarrhea, azotemia, bleeding of the stomach, intestinal bleeding, canker sores, decreased blood platelets, perforation of the intestine, bacterial infection, inflammation of gums or mouth, inflammation of the stomach lining or intestinal lining, bacterial sepsis, stomach ulcer, intestinal ulcer, sun sensitive skin, dizziness, loss of appetite, low energy, and vomiting. In certain embodiments, intolerance can be determined by the subject or by a medical professional upon examination of the subject. In various embodiments, the DMARD is selected from the group consisting of methotrexate, sulfasalazine, leflunomide, and hydroxychloroquine. In certain embodiments, the DMARD is methotrexate.

In other embodiments, the subject suffers from diminishment in quality of life due to RA. In certain embodiments, subjects suffering from diminishment in quality of life due to RA score as more severe than average on a metric selected from Change From Baseline in European Quality of Life-5 Dimension 3 Level (EQ-5D-3L), Change From Baseline in Rheumatoid Arthritis Impact of Disease (RAID), Work Days Missed Due to Arthritis, Work Productivity Reduced by 50% Due to Arthritis, Rate of Arthritis Interference With Work Productivity, House Work Days Missed Due to Arthritis, Days With Household Work Productivity Reduced by 50% Due to Arthritis, Days With Family/Social/Leisure Activities Missed Due to Arthritis, Days With Outside Help Hired Due to Arthritis, Rate of RA Interference With Household Work Productivity, Morning Stiffness VAS, Individual ACR Component—TJC and SJC, Individual ACR Component—Physician Global VAS, Participant Global VAS and Pain VAS, and Individual ACR Component—ESR Level. In other embodiments, the subject has a more severe than average HAQ-DI or DAS-28 score before starting treatment.

In certain embodiments, subjects who score as more severe than average on one or more metrics have a score that is more severe than the baseline value for the metric listed in one or more of Tables 2, 3, 5, or 8, below. In various embodiments, a subject having a score of baseline value or more severe than baseline value on one or more metrics listed in one or more of Tables 2, 3, 5, or 8, below, after receiving treatment indicates that the subject is an inadequate responder to the treatment. In other embodiments, a subject having a score more severe than baseline value on one or more metrics listed in one or more of Tables 2, 3, 5, or 8, below, after receiving treatment indicates that the subject is an inadequate responder to the treatment.

In certain embodiments, the subjects who have scores for metrics that are more severe than the baseline value for the metric listed in one or more of Tables 2, 3, 5, or 8 have scores that are at least 10, 20, 30, 40, 50, 60, 70, 80, 90 or 100% more severe than baseline.

All publications mentioned herein are incorporated herein by reference in their entirety for all purposes.

### Example: A Randomized, Double-Blind, Parallel-Group Study Assessing the Efficacy and Safety of Sarilumab Monotherapy Versus Adalimumab Monotherapy in Patients with Rheumatoid Arthritis (Study No. EFC14092, Study Title: SARIL-RA-MONARCH)

### Objectives:

### Primary Objective:

To demonstrate that sarilumab monotherapy is superior to adalimumab monotherapy with respect to signs and symptoms as assessed by the DAS28-ESR at Week 24 in patients with active RA who are either intolerant of, or considered inappropriate candidates for, continued treatment with methotrexate (MTX); or, after at least 12 weeks of continuous treatment with MTX, are determined to be inadequate responders.

### Secondary Objectives:

To demonstrate that sarilumab monotherapy is superior to adalimumab monotherapy in patients with active RA who are either intolerant of, or considered inappropriate candidates for, continued treatment with MTX; or, after at least 12 weeks of continuous treatment with MTX, are determined to be inadequate responders, with respect to:
reduction of signs and symptoms of RA at Week 24
improvement in quality of life as measured by patient reported outcomes questionnaires at Week 24

To assess the safety and tolerability of sarilumab monotherapy (including immunogenicity) throughout the study.

### Methodology:

Randomized, double-blind, double-dummy, parallel-group study for 24 weeks followed by open-label sarilumab treatment. Randomization was stratified by regions.

Number of patients:

### Planned: 340

### Randomized: 369

### Treated: 368

### Evaluated: Efficacy: 369

Safety: 368

### Diagnosis and Criteria for Inclusion:

Patients with active RA for ≥3 months who were either intolerant of, or considered inappropriate candidates for, continued treatment with MTX; or, after at least 12 weeks of continuous treatment with MTX, are determined to be inadequate responders.

### Study Treatments

Investigational medicinal product(s): sarilumab 200 mg q2w or placebo and adalimumab 40 mg q2w or placebo in prefilled syringes for subcutaneous administration.

Duration of treatment: 24 Weeks of randomized treatment

Duration of observation: Up to 34 weeks for the randomized period (4 week screening, 24 weeks of treatment, and 6 week posttreatment observation if patient doesn't enter the open-label extension)

### Criteria for Evaluation:

### Efficacy:

### Primary Endpoint:

Change from baseline in DAS28-ESR at Week 24

### Secondary Endpoints:

DAS28-ESR (remission)—Week 24
ACR50 response—Week 24
ACR70 response—Week 24
ACR20 response—Week 24

### HAQ-DI—Week 24

### SF-36 Physical—Week 24

### FACIT Fatigue—Week 24

### SF-36 Mental—Week 24

### Safety:

Adverse events reported by the patient or noted by the Investigator, adjudicated cardiovascular events and standard hematology and blood chemistry, electrocardiogram (ECG), physical examinations, and occurrence of anti-drug antibodies to sarilumab.

### Statistical Methods:

The efficacy analysis population is the intent-to-treat (ITT) population, which includes all randomized subjects. For efficacy analysis, subjects were analyzed in the treatment group to which they were randomized, irrespective of the treatment they actually received. The primary safety analysis was conducted on all randomized patients who were exposed to at least one injection of the study medication. Subjects are analyzed in the treatment group that they actually received, irrespective of which group they were randomized to. For the primary efficacy, the change from baseline in the DAS28-ESR at Week 24, data collected on or before Week 24 including after adalimumab (or matching placebo) dose increase were used. Data collected after treatment discontinuation were set to missing. No imputation was performed. The primary efficacy endpoint was assessed by using a mixed model for repeated measures (MMRM) with the baseline covariate and factors for treatment, region, visit and visit by treatment interaction.

Safety summaries were descriptive and no hypothesis testing was conducted. Summary of treatment emergent adverse events (TEAE) was based on MedDRA coding of verbatim terms reported by investigators. TEAEs were defined as any adverse events that newly developed or worsened or became serious on or after the day of first dose intake of investigational medicinal product (IMP), up to the day of end of study or until start of extension treatment. For selected laboratory tests, vital signs and ECGs, incidences of potentially clinically significant abnormality (PCSA) values were summarized.

### Population Characteristics:

Three hundred sixty-nine (369) patients represented the ITT population. Three hundred sixty-eight (368) patients represented the safety population. It was determined that 321 (87%) patients successfully completed the 24-week treatment period, of which 320 patients continued in the open-label extension period to continue long term treatment with sarilumab. 47 (12.7%) patients permanently discontinued the double-blind study treatment, of which 17 patients continued follow-up visit to Week 24. The demographic and disease characteristics at baseline were generally similar between treatment groups (see Table 2 showing baseline values). Patients on sarilumab tended to be younger with a longer duration of RA and lower baseline CRP compared to adalimumab.

The mean duration of study treatment was 158 days in the sarilumab group and 154 days in the adalimumab group. The percentage of patients with IMP compliance≥80% was 99% and 100%, respectively.

TABLE 2
Baseline values for the subjects

| | | Adalimumab | Sarilumab | |
|---|---|---|---|---|
| | | 40 mg q2w | 200 mg q2w | All |
| EQ-5D-3L, single index utility | n | 183 | 182 | 365 |
| | Mean (SD) | 0.30 (0.34) | 0.31 (0.33) | 0.31 (0.33) |
| | Median | 0.52 | 0.52 | 0.52 |
| | Min:Max | −0.6:0.9 | −0.3:0.8 | −0.6:0.9 |
| EQ-5D-3L, VAS | n | 183 | 183 | 366 |
| | Mean (SD) | 41.33 (19.26) | 42.52 (22.13) | 41.93 (20.72) |
| | Median | 40.00 | 40.00 | 40.00 |
| | Min:Max | 0.0:90.0 | 0.0:98.0 | 0.0:98.0 |
| RAID | n | 185 | 183 | 368 |
| | Mean (SD) | 6.40 (2.03) | 6.69 (1.72) | 6.54 (1.89) |
| | Median | 6.52 | 6.81 | 6.67 |
| | Min:Max | 0.9:10.0 | 1.3:10.0 | 0.9:10.0 |
| Work days missed due to arthritis | n | 69 | 79 | 148 |
| | Mean (SD) | 2.04 (5.11) | 2.47 (5.26) | 2.27 (5.18) |
| | Median | 0.00 | 0.00 | 0.00 |
| | Min:Max | 0.0:30.0 | 0.0:30.0 | 0.0:30.0 |
| Days with work productivity reduced | n | 69 | 78 | 147 |
| by >=50% due to arthritis | Mean (SD) | 4.90 (7.66) | 5.78 (7.03) | 5.37 (7.32) |
| | Median | 0.00 | 3.50 | 2.00 |
| | Min:Max | 0.0:30.0 | 0.0:28.0 | 0.0:30.0 |
| Rate of arthritis interference with work | n | 69 | 78 | 147 |
| productivity (0-10) | Mean (SD) | 4.86 (3.04) | 5.59 (2.61) | 5.24 (2.83) |
| | Median | 5.00 | 6.00 | 5.00 |
| | Min:Max | 0.0:10.0 | 0.0:10.0 | 0.0:10.0 |
| House work days missed due to | n | 183 | 183 | 366 |
| arthritis | Mean (SD) | 7.33 (9.06) | 8.73 (8.14) | 8.03 (8.63) |
| | Median | 4.00 | 7.00 | 5.00 |
| | Min:Max | 0.0:30.0 | 0.0:30.0 | 0.0:30.0 |
| Days with household work productivity | n | 183 | 183 | 366 |
| reduced by >=50% due to arthritis | Mean (SD) | 9.35 (9.68) | 9.95 (8.78) | 9.65 (9.24) |
| | Median | 5.00 | 8.00 | 7.00 |
| | Min:Max | 0.0:30.0 | 0.0:30.0 | 0.0:30.0 |
| Days with family, social, or leisure | n | 183 | 183 | 366 |
| activities missed due to arthritis | Mean (SD) | 5.56 (8.51) | 5.42 (7.84) | 5.49 (8.17) |
| | Median | 2.00 | 2.00 | 2.00 |
| | Min:Max | 0.0:30.0 | 0.0:30.0 | 0.0:30.0 |
| Days with outside help hired due to | n | 183 | 182 | 365 |
| arthritis | Mean (SD) | 4.60 (8.57) | 5.15 (8.43) | 4.87 (8.49) |
| | Median | 0.00 | 0.00 | 0.00 |
| | Min:Max | 0.0:30.0 | 0.0:30.0 | 0.0:30.0 |
| Rate of arthritis interference with | n | 183 | 182 | 365 |
| household work productivity (0-10) | Mean (SD) | 6.21 (2.93) | 6.58 (2.53) | 6.39 (2.74) |
| | Median | 7.00 | 7.00 | 7.00 |
| | Min:Max | 0.0:10.0 | 0.0:10.0 | 0.0:10.0 |
| Morning stiffness VAS | n | 184 | 183 | 367 |
| | Mean (SD) | 68.02 (21.37) | 70.83 (18.99) | 69.42 (20.24) |
| | Median | 71.00 | 74.00 | 72.00 |
| | Min:Max | 10.0:100.0 | 3.0:100.0 | 3.0:100.0 |
| Tender joint count | n | 185 | 184 | 369 |
| | Mean (SD) | 26.68 (13.63) | 27.96 (13.19) | 27.32 (13.41) |
| | Median | 24.00 | 25.00 | 24.00 |
| | Min:Max | 7.0:68.0 | 6.0:64.0 | 6.0:68.0 |
| Swollen joint count | n | 185 | 184 | 369 |
| | Mean (SD) | 17.51 (10.25) | 18.57 (10.74) | 18.04 (10.50) |
| | Median | 15.00 | 16.00 | 15.00 |
| | Min:Max | 1.0:66.0 | 6.0:61.0 | 1.0:66.0 |
| Physician global VAS | n | 185 | 184 | 369 |
| | Mean (SD) | 65.95 (17.07) | 66.33 (15.72) | 66.14 (16.39) |
| | Median | 68.00 | 67.00 | 68.00 |
| | Min:Max | 12.0:100.0 | 10.0:100.0 | 10.0:100.0 |
| Patient global VAS | n | 185 | 184 | 369 |
| | Mean (SD) | 67.84 (18.41) | 68.04 (17.49) | 67.94 (17.93) |
| | Median | 70.00 | 70.00 | 70.00 |
| | Min:Max | 14.0:100.0 | 1.0:100.0 | 1.0:100.0 |
| Pain VAS | n | 185 | 184 | 369 |
| | Mean (SD) | 71.44 (18.96) | 71.58 (18.65) | 71.51 (18.78) |
| | Median | 76.00 | 76.00 | 76.00 |
| | Min:Max | 14.0:100.0 | 7.0:100.0 | 7.0:100.0 |
| CRP (mg/dL) | n | 185 | 184 | 369 |
| | Mean (SD) | 2.41 (3.10) | 1.74 (2.13) | 2.07 (2.68) |
| | Median | 0.96 | 0.80 | 0.89 |
| | Min:Max | 0.0:20.2 | 0.0:12.0 | 0.0:20.2 |
| ESR (mm/h) | n | 185 | 184 | 369 |
| | Mean (SD) | 47.51 (23.23) | 46.48 (21.75) | 47.00 (22.48) |
| | Median | 40.00 | 39.00 | 39.00 |
| | Min:Max | 7.0:130.0 | 4.0:120.0 | 4.0:130.0 |

### Efficacy Results:

### Primary Endpoint

The change in the DAS28-ESR (Disease Activity Score 28—erythrocyte sedimentation rate) score from baseline to Week 24 showed a significantly greater decrease in the sarilumab group compared to adalimumab (with a mean difference of −1.077 units, p-value <0.0001, Table 3). This effect was seen as early as Week 12. Both planned sensitivity analyses confirmed these data.

TABLE 3

| | Adalimumab 40 mg q2w | | Sarilumab 200 mg q2w | |
|---|---|---|---|---|
| DAS28-ESR | (N = 185) | | (N = 184) | |
| Number | 163 | | 165 | |
| Baseline Mean (SD) | 6.73 | (0.83) | 6.81 | (0.76) |
| Week 24 Mean (SD) | 4.51 | (1.35) | 3.46 | (1.44) |
| Change Mean (SD) | −2.22 | (1.36) | −3.35 | (1.37) |
| LS mean (SE) | −2.20 | (0.106) | −3.28 | (0.105) |
| LS mean diff, 95% CI | | | −1.077 | (−1.361, −0.793) |

| | |
|---|---|
| P-value vs Adalimumab^{a} | <0.0001 |

DAS28-ESR = 0.56 × sqrt(28TJC) + 0.28 × sqrt(28SJC) + 0.70 × Ln(ESR) + 0.014 × Patient global VAS.
[sqrt = square root].
All assessments are set to missing from the time a patient prematurely discontinues study medication.
Note:
Number = Number of patients with assessment at both baseline and Week 24.
LS, least squares; SD, standard deviation; SE, standard error; CI, confidence interval.
^{a}Type III sum of squares MMRM with PROC MIXED assuming an unstructured covariance structure: model = baseline, treatment, region, visit, and treatment-by-visit interaction.

### Secondary Endpoints

Table 4 shows the results for the pre-specified hierarchy of primary and secondary efficacy endpoints including assessments of quality of life/physical function. The results that are bolded are statistically significant according to the procedure of analysis. The last statistically significant endpoint in the testing hierarchy was the SF-36 physical score.

TABLE 4

| | Adalimumab | | Sarilumab | | | |
|---|---|---|---|---|---|---|
| | 40 mg q2w | | 200 mg q2w | | | |
| | (N = 185) | | (N = 184) | | | |
| Parameter | Estimate^{a} | | | | P-value^{b} | Delta |
| Primary endpoint | | | | | | |
| DAS28-ESR | −2.20 | (0.106) | −3.28 | (0.105) | <0.0001 | −1.08 |
| Secondary endpoints | | | | | | |
| DAS28-ESR remission | 13 | (7.0%) | 49 | (26.6%) | <0.0001 | 19.6% |
| (<2.6) - Week 24 | | | | | | |
| ACR50 response -Week 24 | 55 | (29.7%) | 84 | (45.7%) | 0.0017 | 16.0% |
| ACR70 response -Week 24 | 22 | (11.9%) | 43 | (23.4%) | 0.0036 | 11.5% |
| ACR20 response -Week 24 | 108 | (58.4%) | 132 | (71.7%) | 0.0074 | 13.3% |
| HAQ-DI - Week 24 | −0.43 | (0.045) | −0.61 | (0.045) | 0.0037 | −0.18 |
| SF-36 Physical - Week 24 | 6.09 | (0.555) | 8.74 | (0.555) | 0.0006 | 2.65 |
| FACIT Fatigue - Week 24 | 8.41 | (0.709) | 10.18 | (0.701) | 0.0689 | 1.77 |
| SF-36 Mental - Week 24 | 6.83 | (0.774) | 7.86 | (0.773) | 0.3319 | 1.03 |

^{a}Values presented are number and percent of responders for binary variables and LS mean change from baseline with standard error for continuous variables
^{b}Nominal p-values.
All values in bold font are significant according to the hierarchical testing procedure.

As can be seen in Table 4, the number of patients achieving a DAS28-ESR remission (<2.6) at Week 24 was much higher in the sarilumab group vs. adalimumab group (26.6% of patients vs. 7% of patients, p-value<0.0001).

In addition, data were obtained that showed that the number of patients achieving a Low disease activity (DAS28-ESR<3.2) at Week 24 was also much higher in the sarilumab group vs. adalimumab group (42.9% of patients vs. 14.1% of patients, p-value<0.0001).

In addition, and as can be seen from Table 4:
the number of patients achieving a ACR20 response at Week 24 was much higher in the sarilumab group vs. adalimumab group (71.7% of patients vs. 58.4% of patients, p-value<0.008),
the number of patients achieving a ACR50 response at Week 24 was much higher in the sarilumab group vs. adalimumab group (45.7% of patients vs. 29.7% of patients, p-value<0.002),
the number of patients achieving a ACR70 response at Week 24 was much higher in the sarilumab group vs. adalimumab group (23.4% of patients vs. 11.9% of patients, p-value<0.004).
   Concerning the physical function assessment (HAQ-DI scores), a more detailed analysis is provided in Tables 5, 6 and 7. Table 4 and 5 show that the LS mean change from baseline in HAQ-DI was 0.61 in the sarilumab group vs. 0.43 in the adalimumab group (p-value<0.004).

TABLE 5
Change from baseline in HAQ-DI at Week 24 - ITT population

| | | |
|---|---|---|
| | Adalimumab 40 mg q2w | Sarilumab 200 mg q2w |
| HAQ-DI | (N = 185) | (N = 184) |

| | | |
|---|---|---|
| Number | 158 | 165 |

| Baseline Mean (SD) | 1.62 | (0.64) | 1.64 | (0.54) |
|---|---|---|---|---|
| Week 24 Mean (SD) | 1.21 | (0.66) | 1.01 | (0.65) |
| Change Mean (SD) | −0.42 | (0.58) | −0.63 | (0.66) |
| LS mean (SE) | −0.43 | (0.045) | −0.61 | (0.045) |
| LS mean diff, 95% CI | | | −0.182 | (−0.305, −0.059) |
| P-value vs | | | 0.0037 | |
| Adalimumab | | | | |

LS, least squares; SD, standard deviation; SE, standard error; CI, confidence interval

TABLE 6

| HAQDI Responder | Adalimumab 40 mg q2w | Sarilumab 200 mg q2w | |
|---|---|---|---|
| (≥0.3) Week 24 n(%) | (N = 185) | (N = 184) | |
| Number | 185 | 184 | |
| Responders | 88 (47.6%) | 114 | (62.0%) |
| Non-responders | 97 (52.4%) | 70 | (38.0%) |
| P-value vs Adalimumab | | 0.0057 | |
| OR, CI vs Adalimumab | | 1.785 | (1.180, 2.698) |

Table 6 shows that the number of patients achieving a change from baseline ≥0.3 for the HAQ-DI was much higher in the sarilumab group vs. adalimumab group (62% vs. 47.6% of patients, p-value<0.006).

TABLE 7

| HAQDI Responder | Adalimumab 40 mg q2w | Sarilumab 200 mg q2w | |
|---|---|---|---|
| (≥0.22) Week 24 n(%) | (N = 185) | (N = 184) | |
| Number | 185 | 184 | |
| Responders | 100 (54.1%) | 124 | (67.4%) |
| Non-responders | 85 (45.9%) | 60 | (32.6%) |
| P-value vs Adalimumab | | 0.0090 | |
| OR, CI vs Adalimumab | | 1.747 | (1.147, 2.663) |

Table 7 shows that the number of patients achieving a change from baseline ≥0.22 for the HAQ-DI was much higher in the sarilumab group vs. adalimumab group (67.4% vs. 54.1% of patients, p-value<0.009).

In addition, Table 8 shows that the change in the DAS28-CRP (Disease Activity Score 28—C reactive protein) score from baseline to Week 24 showed a significantly greater decrease in the sarilumab group compared to adalimumab (with a mean difference of −0.884 unit, p-value <0.0001).

TABLE 8

| | Adalimumab 40 mg q2w | Sarilumab 200 mg q2w | |
|---|---|---|---|
| DAS28-CRP | (N=185) | (N = 184) | |
| Number | 156 | 163 | |
| Baseline Mean (SD) | 5.98 (0.88) | 6.00 | (0.87) |
| Week 24 Mean (SD) | 3.92 (1.24) | 3.07 | (1.21) |
| Change Mean (SD) | −2.06 (1.22) | −2.93 | (1.25) |
| LS mean (SE) | −1.97 (0.094) | −2.86 | (0.093) |
| LS mean diff, 95% CI | | −0.884 | (−1.138, −0.629) |

| | |
|---|---|
| P-value vs Adalimumab^{a} | <0.0001 |

All assessments are set to missing from the time a patient prematurely discontinues study medication.
LS, least squares; SD, standard deviation; SE, standard error; CI, confidence interval.
DAS28-CRP = 0.56 × sqrt(28TJC) + 0.28 × sqrt(28SJC) + 0.36 × Ln [CRP(mg/L) + 1] + 0.014 × GH(VAS) + 0.96

The number of patients achieving a DAS28-CRP remission (<2.6) at Week 24 was also much higher in the sarilumab group vs. adalimumab group (34.2% of patients vs. 13.5% of patients, p-value<0.0001).

Table 9 shows that the patients in the sarilumab group were also twice as likely to achieve Clinical Disease Activity Index (CDAI) remission (CDAI≤2.8) at week 24 vs adalimumab (P<0.05).

TABLE 9

| CDAI remission | | | |
|---|---|---|---|
| (CDAI ≤ 2.8) at | Adalimumab 40 mg q2w | Sarilumab 200 mg q2w | |
| Week 24 n(%) | (N = 185) | (N = 184) | |
| Number | 185 | 184 | |
| Yes | 5 (2.7%) | 13 | (7.1%) |
| No | 180 (97.3%) | 171 | (92.9%) |
| P-value vs Adalimumab ^{a} | | 0.0468 | |
| OR, CI vs Adalimumab ^{b} | | 2.869 | (0.981, 8.389) |

The CDAI is a composite index constructed to measure clinical remission in RA that does not include a laboratory test, and is a numerical summation of four components: SJC (28 joints), tender joint count (28 joints), patient's global disease activity (in cm), and physician's global assessment (in cm). Scores may range from 0 to 76. See Aletaha, D and Smolen J. The Simplified Disease Activity Index (SDAI) and the Clinical Disease Activity Index (CDAI): A review of their usefulness and validity in rheumatoid arthritis, Clin Exp Rheumatol 2005; 23 (Suppl.39): 5100-5108, incorporated herein by reference in its entirety.

In addition, Table 10 shows that the change in the CDAI score from baseline to Week 24 showed a significantly greater decrease in the sarilumab group compared to adalimumab (with a mean difference of −3.741 unit, p-value <0.002).

TABLE 10

| Change from baseline | Adalimumab 40 mg q2w | Sarilumab 200 mg q2w | |
|---|---|---|---|
| in CDAI at Week 24 | (N = 185) | (N = 184) | |
| CDAI (0-76) | | | |
| LS mean (SE) | −25.20 (0.842) | −28.94 | (0.834) |
| LS mean diff, 95% CI | | −3.741 | (−6.016, −1.466) |

| | |
|---|---|
| P-value vs adalimumab | 0.0013 |

CDAI = 28TJC + 28SJC + Patient global VAS + Physician global VAS.
All assessments are set to missing from the time a patient receives rescue medication or discontinues study medication early. No imputation is performed.

EQ-5D-3L:

| | |
|---|---|
| Measure Title | Change From Baseline in European Quality of |
| | Life-5 Dimension 3 Level (EQ-5D-3L) Scores at |
| | Week 24 |
| Measure Description | EQ-5D-3L is a standardized, generic measure |
| | of health outcome. EQ-5D was designed for |
| | self-completion by participants. EQ-5D |
| | specifically included to address concerns |
| | regarding the health economic impact of RA. |
| | EQ-5D-3L comprises of 5 questions on mobility, |
| | self-care, pain/discomfort, usual activities, and |
| | psychological status with 3 possible answers for |
| | each item (1 = no problem, 2 = moderate |
| | problems, 3 = severe problems). The 5- |
| | dimensional 3-level systems are converted into |
| | a single index utility score between 0 to 1, |
| | where higher score indicates a better health |
| | state. EQ-5D-3L-VAS records the participant's |
| | self-rated health on a vertical VAS that allows |
| | the participants to indicate their health state that |
| | can range from 0 (worst imaginable) to 100 |
| | (best imaginable). LS mean and SE at Week 24 |
| | were obtained using MMRM approach. |
| Time Frame | Baseline, Week 24 |
| Safety Issue? | No |

### Analysis Population Description

ITT population. Number of participants analyzed=participants with EQ-5D-3L score assessments both at baseline and Week 24. Here ‘n’ signifies number of participants with available data for specified category.

Measured Values

| | Adalimumab 40 mg | Sarilumab 200 mg |
|---|---|---|
| Number of Participants | 156 | 164 |
| Analyzed | | |
| Change From Baseline in | | |
| European Quality of Life-5 | | |
| Dimension 3 Level (EQ- | | |
| 5D-3L) Scores at Week 24 | | |
| [units: units on a scale] | | |
| Least Squares Mean | | |
| (Standard Error) | | |
| EQ-5D Single index | 0.26 (0.019) | 0.32 (0.019) |
| utility score (n = 156,160) | | |
| EQ-5D VAS | 19.94 (1.720) | 24.22 (1.686) |
| (n = 156,164) | | |

RAID:

| | |
|---|---|
| Measure Title | Change From Baseline in Rheumatoid Arthritis |
| | Impact of Disease (RAID) at Week 24 |
| Measure Description | RAID is a composite measure of the impact of |
| | RA on participants that takes into account 7 |
| | domains: pain, functional disability, fatigue, |
| | physical and emotional well-being, quality of |
| | sleep, and coping. The RAID is calculated |
| | based on 7 numerical rating scales (NRS) |
| | questions. Each NRS is assessed as a number |
| | between 0 and 10 that corresponds to the 7 |
| | domains. The values for each of these domains |
| | are weighed by participant assessment of |
| | relative importance and combined in a single |
| | and calculated with a total score range of 0 (not |
| | affected, very good) to 10 (most affected). LS |
| | mean and SE at Week 24 were obtained using |
| | MMRM approach. |
| Time Frame | Baseline, Week 24 |
| Safety Issue? | Yes |

### Analysis Population Description

ITT population. Number of participants analyzed=participants with RAID assessments both at baseline and Week 24.

Measured Values

| | Adalimumab 40 mg | Sarilumab 200 mg |
|---|---|---|
| Number of Participants | 157 | 161 |
| Analyzed | | |
| Change From Baseline in | −2.30 (0.168) | −3.08 (0.168) |
| Rheumatoid Arthritis | | |
| Impact of Disease (RAID) | | |
| at Week 24 | | |
| [units: units on a scale] | | |
| Least Squares Mean | | |
| (Standard Error) | | |

WPS-RA: Work Days Missed Due to Arthritis

| | |
|---|---|
| Measure Title | Change From Baseline in Work Productivity |
| | Survey - Rheumatoid Arthritis (WPS-RA) at |
| | Week 24: Work Days Missed Due to Arthritis |
| Measure Description | The WPS-RA is a validated questionnaire that |
| | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). Number of work |
| | days missed in the last month by the participant |
| | was reported. LS mean and SE at Week 24 |
| | were obtained using MMRM approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | Adalimumab 40 mg | Sarilumab 200 mg |
|---|---|---|
| Number of Participants | 60 | 70 |
| Analyzed | | |
| Change From Baseline in | 0.05 (0.611) | −0.28 (0.547) |
| Work Productivity Survey - | | |
| Rheumatoid Arthritis (WPS- | | |
| RA) at Week 24: Work Days | | |
| Missed Due to Arthritis | | |
| [units: days] | | |
| Least Squares Mean | | |
| (Standard Error) | | |

WPS-RA: Days With Work Productivity Reduced by ≥50% Due
to Arthritis

| | |
|---|---|
| Measure Title | Change From Baseline in WPS-RA at Week 24: |
| | Days With Work Productivity Reduced by ≥50% Due |
| | to Arthritis |
| Measure | The WPS-RA is a validated questionnaire that |
| Description | evaluates productivity limitations within work and |
| | within home associated with RA over the previous |
| | month. The questionnaire is interviewer-administered |
| | and based on participant self-report. It contains 9 |
| | questions addressing employment status (1 item), |
| | productivity at work (3 items), and within and outside |
| | the home (5 items). Number of work days with |
| | reduced productivity by ≥50% in the last month by |
| | the participants was reported. LS mean and SE at |
| | Week 24 were obtained using MMRM approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | | Sarilumab |
|---|---|---|
| | Adalimumab 40 mg | 200 mg |
| Number of Participants | 60 | 70 |
| Analyzed | | |
| Change From Baseline in | −3.50 (0.525) | −3.74 (0.456) |
| WPS-RA at Week 24: | | |
| Days With Work | | |
| Productivity Reduced by ≥50% | | |
| Due to Arthritis | | |
| [units: days] | | |
| Least Squares Mean | | |
| (Standard Error) | | |

WPS-RA: Rate of Arthritis Interference With Work Productivity

| | |
|---|---|
| Measure Title | Change From Baseline in WPS-RA at Week 24: |
| | Rate of Arthritis Interference With Work |
| | Productivity |
| Measure Description | The WPS-RA is a validated questionnaire that |
| | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). Interference in the |
| | last month with work productivity was measured |
| | on a scale that ranges from 0 (no interference) |
| | to 10 (complete interference). LS mean and SE |
| | at Week 24 were obtained using MMRM |
| | approach. |
| Time Frame | Baseline, Week 24 |
| Safety Issue? | No |

### Analysis Population Description

ITT Population. Number of participants analyzed=participants with WPS-RA: Individual items assessments both at baseline and Week 24.

Measured Values

| | Adalimumab 40 mg | Sarilumab 200 mg |
|---|---|---|
| Number of Participants | 60 | 69 |
| Analyzed | | |
| Change From Baseline in | −25.10 (3.470) | −29.19 (3.073) |
| WPS-RA at Week 24: | | |
| Rate of Arthritis | | |
| Interference With Work | | |
| Productivity | | |
| [units: units on a scale] | | |
| Least Squares Mean | | |
| (Standard Error) | | |

WPS-RA: House Work Days Missed Due to Arthritis

| | |
|---|---|
| Measure Title | Change From Baseline in WPS-RA at Week 24: |
| | House Work Days Missed Due to Arthritis |
| Measure Description | The WPS-RA is a validated questionnaire that |
| | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). Number of days |
| | with no household work in the last month by the |
| | participants was reported. LS mean and SE at |
| | Week 24 were obtained using MMRM |
| | approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | | Sarilumab |
|---|---|---|
| | Adalimumab 40 mg | 200 mg |
| Number of Participants Analyzed | 163 | 169 |
| Change From Baseline in WPS-RA | −4.22 (0.405) | −5.49 (0.400) |
| at Week 24: House Work Days | | |
| Missed Due to Arthritis | | |
| [units: days] | | |
| Least Squares Mean (Standard | | |
| Error) | | |

WPS-RA: Days With Household Work Productivity Reduced by ≥50%
Due to Arthritis

| | |
|---|---|
| Measure Title | Change From Baseline in WPS-RA at Week 24: |
| | Days With Household Work Productivity |
| | Reduced by ≥50% Due to Arthritis |
| Measure Description | The WPS-RA is a validated questionnaire that |
| | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). Number of days |
| | with reduced household work productivity by ≥50% |
| | in the last month by the participants was |
| | reported. LS mean and SE at Week 24 were |
| | obtained using MMRM approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | Adalimumab 40 mg | Sarilumab 200 mg |
|---|---|---|
| Number of Participants | 163 | 169 |
| Analyzed | | |
| Change From Baseline in | −4.87 (0.451) | −6.70 (0.445) |
| WPS-RA at Week 24: | | |
| Days With Household | | |
| Work Productivity | | |
| Reduced by ≥50% Due to | | |
| Arthritis | | |
| [units: days] | | |
| Least Squares Mean | | |
| (Standard Error) | | |

WPS-RA: Days With Family/Social/Leisure Activities Missed
Due to Arthritis

| | |
|---|---|
| Measure Title | Change From Baseline in WPS-RA at Week 24: |
| | Days With Family/Social/Leisure Activities |
| | Missed Due to Arthritis |
| Measure Description | The WPS-RA is a validated questionnaire that |
| | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). Number of days |
| | missed of family/social/leisure activities in the |
| | last month in the last month by the participants |
| | was reported. LS mean and SE at Week 24 |
| | were obtained using MMRM approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | Adalimumab 40 mg | Sarilumab 200 mg |
|---|---|---|
| Number of Participants | 163 | 169 |
| Analyzed | | |
| Change From Baseline in | −3.33 (0.376) | −4.14 (0.371) |
| WPS-RA at Week 24: | | |
| Days With | | |
| Family/Social/Leisure | | |
| Activities Missed Due to | | |
| Arthritis | | |
| [units: days] | | |
| Least Squares Mean | | |
| (Standard Error) | | |

WPS-RA: Days With Outside Help Hired Due to Arthritis

| | |
|---|---|
| Measure Title | Change From Baseline in WPS-RA at Week 24: |
| | Days With Outside Help Hired Due to Arthritis |
| Measure | The WPS-RA is a validated questionnaire that |
| Description | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). Number of days |
| | with outside help hired in the last month by the |
| | participant was reported. LS mean and SE at |
| | Week 24 were obtained using MMRM |
| | approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | Adalimumab 40 mg | Sarilumab 200 mg |
|---|---|---|
| Number of Participants | 163 | 168 |
| Analyzed | | |
| Change From Baseline in | −2.57 (0.401) | −3.43 (0.398) |
| WPS-RA at Week 24: | | |
| Days With Outside Help | | |
| Hired Due to Arthritis | | |
| [units: days] | | |
| Least Squares Mean | | |
| (Standard Error) | | |

WPS-RA: Rate of RA Interference
With Household Work Productivity

| | |
|---|---|
| Measure Title | Change From Baseline in WPS-RA at Week 24: |
| | Rate of RA Interference With Household Work |
| | Productivity |
| Measure | The WPS-RA is a validated questionnaire that |
| Description | evaluates productivity limitations within work |
| | and within home associated with RA over the |
| | previous month. The questionnaire is |
| | interviewer-administered and based on |
| | participant self-report. It contains 9 questions |
| | addressing employment status (1 item), |
| | productivity at work (3 items), and within and |
| | outside the home (5 items). The RA interference |
| | in the last month with household work |
| | productivity was measured on a scale that |
| | ranges from 0 (no interference) to 10 (complete |
| | interference). LS mean and SE at Week 24 |
| | were obtained using MMRM approach. |
| Time Frame | Baseline, Week 24 |
| Safety Issue? | No |

### Analysis Population Description

ITT population. Number of participants analyzed=participants with WPS-RA: Individual items assessments both at baseline and Week 24.

Measured Values

| | Adalimumab 40 mg | Sarilumab 200 mg |
|---|---|---|
| Number of Participants | 163 | 168 |
| Analyzed | | |
| Change From Baseline in | −26.05 (2.110) | −32.76 (2.099) |
| WPS-RA at Week 24: | | |
| Rate of RA Interference | | |
| With Household Work | | |
| Productivity | | |
| [units: units on a scale] | | |
| Least Squares Mean | | |
| (Standard Error) | | |

Morning Stiffness VAS:

| | |
|---|---|
| Measure Title | Change From Baseline in Morning Stiffness VAS at |
| | Week 24 |
| Measure | RA is associated with stiffness of joints, especially in |
| Description | the morning after prolonged stationery state. The |
| | degree of stiffness can be an indicator of disease |
| | severity. The severity of morning stiffness was |
| | assessed on a VAS scale from 0 mm (no problem) to |
| | 100 mm (major problem). LS mean and SE at Week |
| | 24 were obtained using MMRM approach. |
| Time Frame | Baseline, Week 24 |
| Safety Issue? | No |

### Analysis Population Description

ITT population. Number of participants analyzed=participants with morning stiffness VAS assessments both at baseline and Week 24.

Measured Values

| | Adalimumab 40 mg | Sarilumab 200 mg |
|---|---|---|
| Number of Participants | 156 | 165 |
| Analyzed | | |
| Change From Baseline in | −29.29 (1.970) | −35.08 (1.947) |
| Morning Stiffness VAS at | | |
| Week 24 | | |
| [units: mm] | | |
| Least Squares Mean | | |
| (Standard Error) | | |

Individual ACR Component - TJC and SJC:

| | |
|---|---|
| Measure Title | Change From Baseline in Individual ACR Component - |
| | TJC and SJC at Week 24 |
| Measure | ACR components were: TJC, SJC, physician global |
| Description | VAS, participant global VAS, pain VAS, HAQ-DI & |
| | acute phase reactant (hs-CRP and ESR levels). 68 |
| | joints were assessed for tenderness (TJC scoring 0- |
| | 68) and 66 joints for swelling (SJC scoring 0-66). The |
| | 66 SJC evaluated the following joints: |
| | temporomandibular, sternoclavicular, |
| | acromioclavicular, shoulder, elbow, wrist, |
| | metacarpophalangeal, interphalangeal of thumb, distal |
| | interphalangeal, proximal interphalangeal, knee, ankle |
| | mortise, ankle tarsus, metatarsophalangeal, |
| | interphalangeal of great toe, and proximal/distal |
| | interphalangeal of the toes. The TJC examined hip |
| | joints, in addition to the joints assessed for SJC. |
| | Increase in number of tender joints/swollen joints |
| | indicated severity. LS mean and SE at Week 24 were |
| | obtained using MMRM approach. |
| Time Frame | Baseline, Week 24 |
| Safety Issue? | No |

### Analysis Population Description

ITT population. Number of participants analyzed=participants with TJC and SJC assessments both at baseline and Week 24.

Measured Values

| | Adalimumab 40 mg | Sarilumab 200 mg |
|---|---|---|
| Number of Participants | 158 | 166 |
| Analyzed | | |
| Change From Baseline in | | |
| Individual ACR | | |
| Component - TJC and | | |
| SJC at Week 24 | | |
| [units: joints] | | |
| Least Squares Mean | | |
| (Standard Error) | | |
| TJC | −16.45 (0.781) | −18.23 (0.772) |
| SJC | −12.20 (0.450) | −13.44 (0.444) |

Individual ACR Component - Physician Global
VAS, Participant Global VAS and Pain VAS:

| | |
|---|---|
| Measure Title | Change From Baseline in Individual ACR Component - |
| | Physician Global VAS, Participant Global VAS and Pain |
| | VAS at Week 24 |
| Measure | ACR components were: TJC, SJC, physician global |
| Description | VAS, participant global VAS, pain VAS, HAQ-DI & |
| | acute phase reactant (hs-CRP and ESR levels). |
| | Physician global VAS & participant global VAS was |
| | done on 100 mm horizontal anchored VAS, ranging |
| | from 0 “no arthritis activity” to 100 |
| | “maximal arthritis activity” and Pain VAS |
| | on 100 mm VAS, ranging from 0 “no pain” to |
| | 100 “worst pain”. LS mean and SE at Week 24 |
| | were obtained using MMRM approach. |
| Time Frame | Baseline, Week 24 |
| Safety Issue? | No |

### Analysis Population Description

Number of participants analyzed=number of participants with individual ACR components assessment at both baseline and specified time points. Here ‘n’ signifies number of participants with available data for specified category.

Measured Values

| | Adalimumab 40 mg | Sarilumab 200 mg |
|---|---|---|
| Number of Participants | 158 | 166 |
| Analyzed | | |
| Change From Baseline in | | |
| Individual ACR Component - | | |
| Physician Global VAS, | | |
| Participant Global VAS and | | |
| Pain VAS at Week 24 | | |
| [units: mm] | | |
| Least Squares Mean | | |
| (Standard Error) | | |
| Physician global VAS | −37.80 (1.431) | −45.33 (1.414) |
| (n = 158, 166) | | |
| Participant global VAS | −24.82 (1.752) | −33.30 (1.731) |
| (n = 158, 165) | | |
| Pain VAS (n = 157, 165) | −27.41 (1.802) | −36.19 (1.776) |

Individual ACR Component - CRP

| | |
|---|---|
| Measure Title | Change From Baseline in Individual ACR |
| | Component - CRP Level at Week 24 |
| Measure | ACR components were: TJC, SJC, physician |
| Description | global VAS, participant global VAS, pain VAS, |
| | HAQ-DI & acute phase reactant (hs-CRP and |
| | ESR levels). An elevated CRP level was |
| | considered a non-specific “marker” for RA. A |
| | reduction level indicates improvement. LS mean |
| | and SE at Week 24 were obtained using MMRM |
| | approach. |
| Time Frame | Baseline, Week 24 |
| Safety Issue? | No |

### Analysis Population Description

ITT population. Number of participants analyzed=participants with CRP assessments both at baseline and Week 24.

Measured Values

| | Adalimumab 40 mg | Sarilumab 200 mg |
|---|---|---|
| Number of Participants | 156 | 164 |
| Analyzed | | |
| Change From Baseline in | −2.91 (1.461) | −17.01 (1.431) |
| Individual ACR Component - | | |
| CRP Level at Week 24 | | |
| [units: mg/L] | | |
| Least Squares Mean | | |
| (Standard Error) | | |

Individual ACR Component- ESR:

| | |
|---|---|
| Measure Title | Change From Baseline in Individual ACR |
| | Component- ESR Level at Week 24 |
| Measure | ACR components were: TJC, SJC, physician |
| Description | global VAS, participant global VAS, pain VAS, |
| | HAQ-DI & acute phase reactant (hs-CRP and |
| | ESR levels). The ESR is a blood test that can |
| | reveal inflammatory activity. Inflammation can |
| | cause the cells to clump together. The farther |
| | the red blood cells have descended, the greater |
| | the inflammatory response. LS mean and SE at |
| | Week 24 were obtained using MMRM |
| | approach. |
| Time Frame | Baseline, Week 24 |
| Safety Issue? | No |

### Analysis Population Description

ITT population. Number of participants analyzed=participants with ESR assessments both at baseline and Week 24.

Measured Values

| | Adalimumab 40 mg | Sarilumab 200 mg |
|---|---|---|
| Number of Participants | 163 | 166 |
| Analyzed | | |
| Change From Baseline in | −12.74 (1.398) | −32.11 (1.388) |
| Individual ACR | | |
| Component- ESR Level at | | |
| Week 24 | | |
| [units: mm/h] | | |
| Least Squares Mean | | |
| (Standard Error) | | |

1-23. (canceled)

24. A method of improving the physical function of a subject suffering from rheumatoid arthritis comprising administering an antibody, wherein:
the antibody comprises the heavy chain variable region comprising the sequence SEQ ID NO:1 and the light chain variable region comprising the sequence SEQ ID NO:2,
the antibody is administered subcutaneously at about 150 mg or about 200 mg once every two weeks to the subject,
the subject is not administered with any other disease-modifying antirheumatic drug (DMARD) in course of administration with the antibody, and
the subject was previously ineffectively treated for rheumatoid arthritis by administering at least one DMARD different from the antibody.

25. The method according to claim 24, wherein the subject achieves after at least 24 weeks of administration of the antibody a change from baseline (BL) in the Health Assessment Questionnaire Disability Index (HAQ-DI) of at least 0.22, particularly of at least 0.30, more particularly of at least 0.60.

26. A method of improving the health related quality of life of a subject suffering from rheumatoid arthritis comprising administering an antibody, wherein:
the antibody comprises a heavy chain variable region comprising sequence SEQ ID NO:1 and a light chain variable region comprising sequence SEQ ID NO:2,
the antibody is administered subcutaneously at about 150 mg or about 200 mg once every two weeks to the subject,
the subject is not administered with any other disease-modifying antirheumatic drug (DMARD) in course of administration with the antibody, and
the subject was previously ineffectively treated for rheumatoid arthritis by administering at least one DMARD different from the antibody.

27. The method according to claim 26, wherein the subject achieves after at least 24 weeks of administration of the antibody a change from baseline (BL) in the Short Form-36 Physical Component Summary (SF-36 PCS) of at least 2.5, particularly of at least 3, more particularly of at least 8.

28. A method of treating rheumatoid arthritis in a subject comprising administering an antibody, wherein:
the antibody comprises a heavy chain variable region comprising sequence SEQ ID NO:1 and a light chain variable region comprising sequence SEQ ID NO:2,
the antibody is administered subcutaneously at about 150 mg or about 200 mg once every two weeks to the subject,
the subject is not administered with any other Disease-Modifying AntiRheumatic Drug (DMARD) in course of administration with the antibody, and
the subject was previously ineffectively treated for rheumatoid arthritis by administering at least one DMARD different from the antibody.

29. The method according to claim 28, wherein the subject achieves after at least 24 weeks of administration of the antibody a 20% improvement in the American College of Rheumatology core set disease index (ACR20), particularly a 50% improvement in the American College of Rheumatology core set disease index (ACR50), more particularly a 70% improvement in the American College of Rheumatology core set disease index (ACR70).

30. The method according to claim 28 or claim 29, wherein the subject achieves after at least 24 weeks of administration of the antibody a change from baseline (BL) in the Disease Activity Score 28—Erythrocyte Sedimentation Rate (DAS28-ESR) of at least 2, particularly of at least 2.5, more particularly of at least 3.

31. The method according to any one of claims 28-30, wherein the subject achieves after at least 24 weeks of administration of the antibody a DAS28-ESR score below 3.2, particularly below 2.6.

32. The method according to any one of claims 24-31, wherein the subject who was previously ineffectively treated for rheumatoid arthritis by administering at least one DMARD different from the antibody, is a subject who was previously ineffectively treated for rheumatoid arthritis by administering methotrexate.

33. The method according to any one claims 24-32, wherein the subject suffering from rheumatoid arthritis is a subject suffering from moderately to severely active rheumatoid arthritis.

34. The method according to any one of claims 24-33, wherein the antibody is administered with a prefilled syringe or with an auto-injector.

35. The antibody for use according to any one of claims 24-34, wherein the antibody is administered as an aqueous buffered solution at about pH 6.0 containing about 21 mM histidine, about 45 mM arginine, about 0.2% (w/v) polysorbate 20, and about 5% (w/v) sucrose.

36. The method according to claim 35, wherein the solution comprises at least about 130 mg/mL of the antibody, particularly the solution comprises about 131.6 mg/mL of the antibody.

37. The method according to claim 35, wherein the solution comprises about 175 mg/mL of the antibody.

38. The method according to any one of claims 24-37, wherein the antibody comprising the heavy chain variable region comprising sequence SEQ ID NO:1 and the light chain variable region comprising sequence SEQ ID NO:2 is sarilumab.

39. The method according to claim 24, wherein the subject intolerant of one or more DMARDs.

40. The method according to claim 39, wherein the DMARD is methotrexate.

41. The method according to claim 24, wherein the subject is considered an inappropriate candidate for continued treatment with one or more DMARDs.

42. The method according to claim 42, wherein the DMARD is methotrexate.

43. The method according to claim 24, wherein the subject suffers from diminishment in quality of life due to RA.

44. The method according to claim 43, wherein the subject scores as more severe than average on a metric selected from Change From Baseline in European Quality of Life-5 Dimension 3 Level (EQ-5D-3L), Change From Baseline in Rheumatoid Arthritis Impact of Disease (RAID), Work Days Missed Due to Arthritis, Work Productivity Reduced by ≥50% Due to Arthritis, Rate of Arthritis Interference With Work Productivity, House Work Days Missed Due to Arthritis, Days With Household Work Productivity Reduced by ≥50% Due to Arthritis, Days With Family/Social/Leisure Activities Missed Due to Arthritis, Days With Outside Help Hired Due to Arthritis, Rate of RA Interference With Household Work Productivity, Morning Stiffness VAS, Individual ACR Component—TJC and SJC, Individual ACR Component—Physician Global VAS, Participant Global VAS and Pain VAS, and Individual ACR Component—ESR Level.

45. The method according to any of the preceding claims 24-44 wherein the antibody is administered subcutaneously at 150 mg once every two weeks to the subject.

46. The method according to any of claims 24-45 wherein the antibody is administered subcutaneously at 200 mg once every two weeks to the subject.

47. (canceled)


SEQUENCE LISTING (10 sequences)

SEQ ID NO: 1 - protein, 116 aa, Artificial Sequence
  Synthetic
  MISC_FEATURE (1)..(116): heavy chain variable region of sarilumab
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 2 - protein, 107 aa, Artificial Sequence
  Synthetic
  MISC_FEATURE (1)..(107): light chain variable region of sarilumab
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 3 - protein, 8 aa, Artificial Sequence
  Synthetic
RFTFDDYA

SEQ ID NO: 4 - protein, 8 aa, Artificial Sequence
  Synthetic
ISWNSGRI

SEQ ID NO: 5 - protein, 9 aa, Artificial Sequence
  Synthetic
AKGRDSFDI

SEQ ID NO: 6 - protein, 6 aa, Artificial Sequence
  Synthetic
QGISSW

SEQ ID NO: 7 - protein, 3 aa, Artificial Sequence
  Synthetic
GAS

SEQ ID NO: 8 - protein, 9 aa, Artificial Sequence
  Synthetic
QQANSFPYT

SEQ ID NO: 9 - protein, 446 aa, Artificial Sequence
  Synthetic - Full Heavy Chain
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Synthetic - Full Light Chain
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC